(19) United States
(12) Patent Application Publication
(10) Pub. No.: US 2018/0014964 A1
(43) Pub. Date: Jan. 18, 2018

(54) Urostomy Bag Carrying System

(71) Applicants: Coleman E. Hill, Winston Salem, NC (US); Mary U. Hill, Winston Salem, NC (US); Christina R. Hill, Winston Salem, NC (US)

(72) Inventors: Coleman E. Hill, Winston Salem, NC (US); Mary U. Hill, Winston Salem, NC (US); Christina R. Hill, Winston Salem, NC (US)

(21) Appl. No.: 15/213,052

(22) Filed: Jul. 18, 2016

(51) Int. Cl. A61F 5/449; A61F 5/448

(52) U.S. Cl. CPC A61F 5/449; A61F 5/448; A61F 2005/4486

(57) ABSTRACT

A urostomy bag carrying system includes a bag that may be carried. The bag has a pair of slots extending into an interior of the bag. A strap is coupled to the bag and the strap may be slung over a shoulder. Thus, the bag may be carried having the bag being positioned to cover a stoma on user. A urostomy bag is selectively positioned within the bag. At least one hose is fluidly coupled to the urostomy bag. The at least one hose is positioned in a selected one of the slots. Thus, the at least one hose may be fluidly coupled to the stoma.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to urostomy bag devices and more particularly pertains to a new urostomy bag device for discretely carrying a urostomy bag and concealing a stoma.

### BRIEF SUMMARY OF THE INVENTION

An embodiment of the disclosure meets the needs presented above by generally comprising a bag that may be carried. The bag has a pair of slots extending into an interior of the bag. A strap is coupled to the bag and the strap may be slung over a shoulder. Thus, the bag may be carried having the bag being positioned to cover a stoma on user. A urostomy bag is selectively positioned within the bag. At least one hose is fluidly coupled to the urostomy bag. The at least one hose is positioned in a selected one of the slots. Thus, the at least one hose may be fluidly coupled to the stoma.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a urostomy bag carrying system according to an embodiment of the disclosure.

FIG. 2 is a front view of an embodiment of the disclosure.

FIG. 3 is a back perspective view of an embodiment of the disclosure.

FIG. 4 is a back view of an embodiment of the disclosure.

FIG. 5 is a top view of an embodiment of the disclosure.

FIG. 6 is a perspective in-use view of an embodiment of the disclosure.

### DETAILED DESCRIPTION OF THE INVENTION

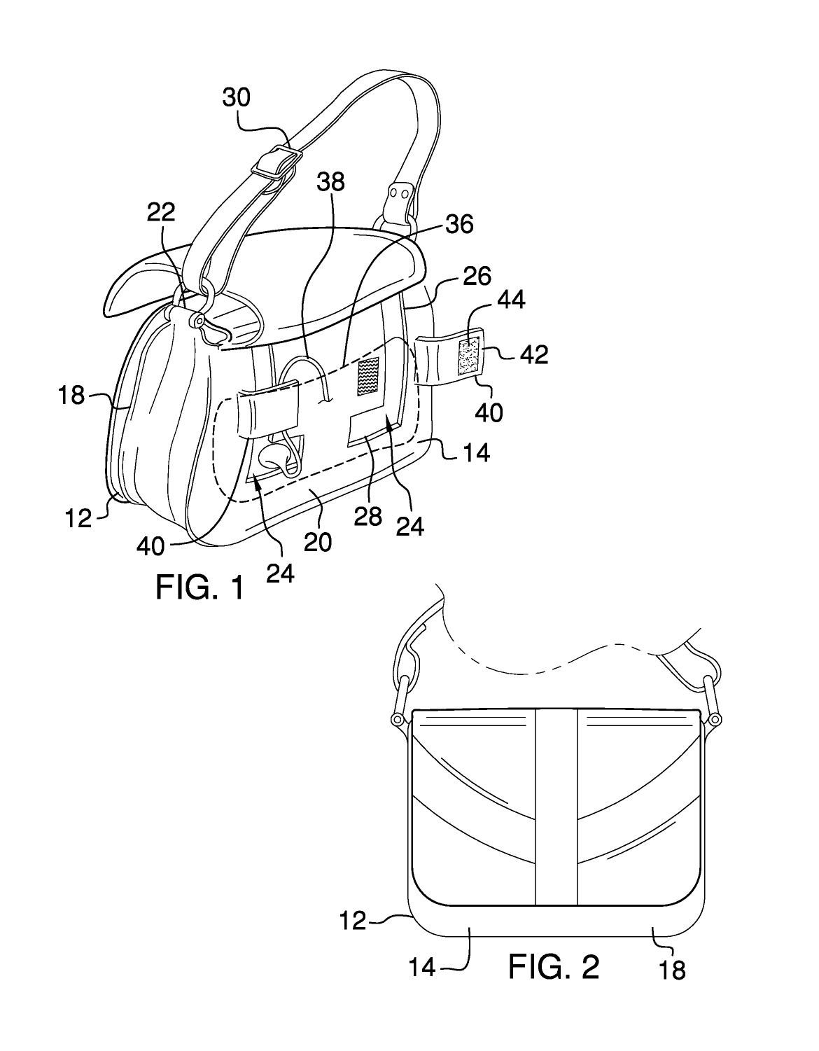

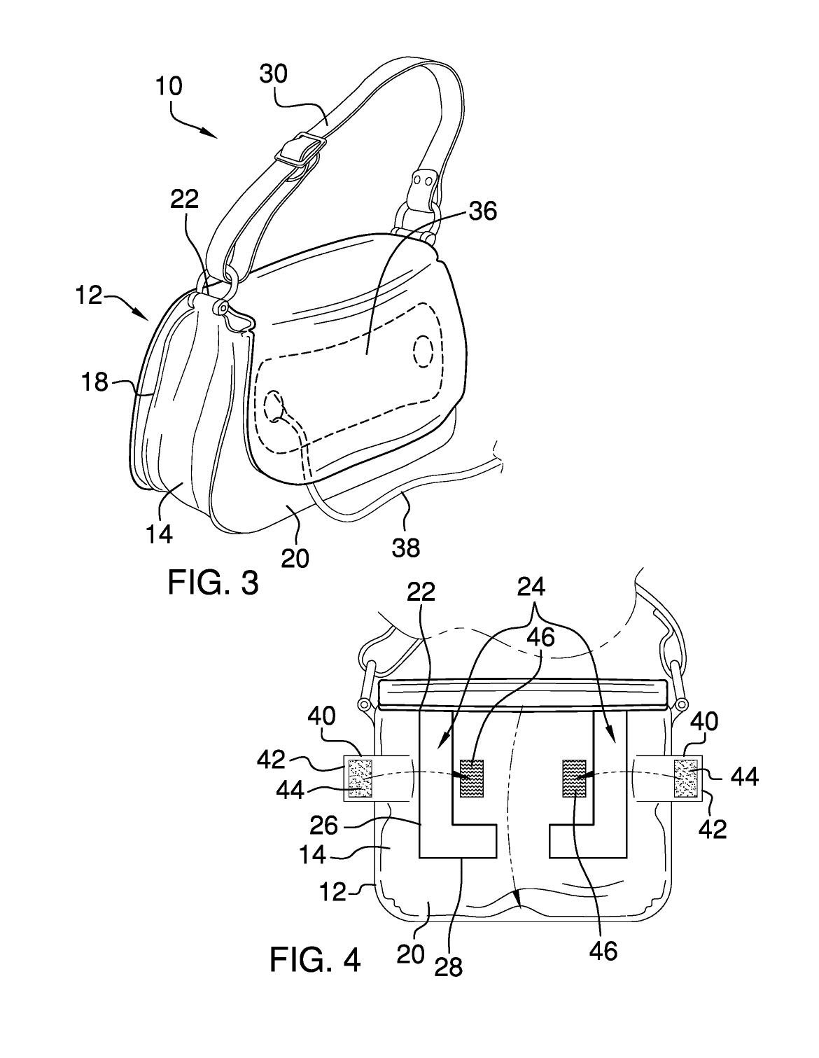

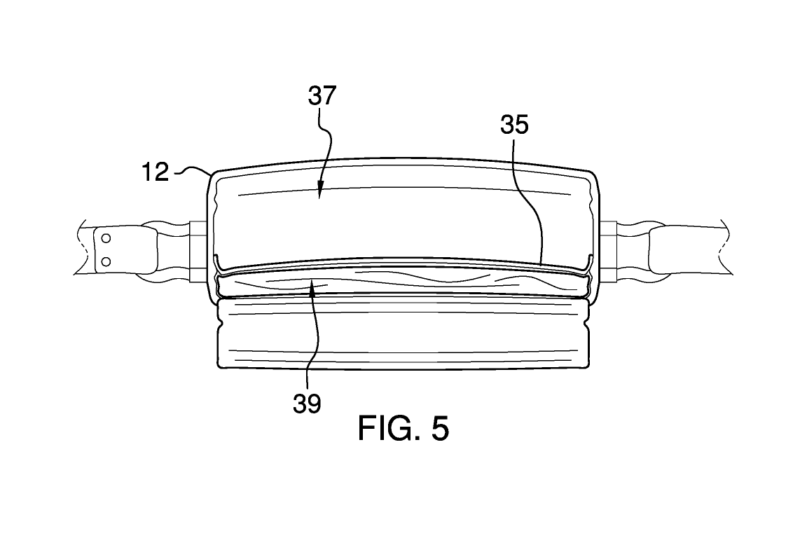

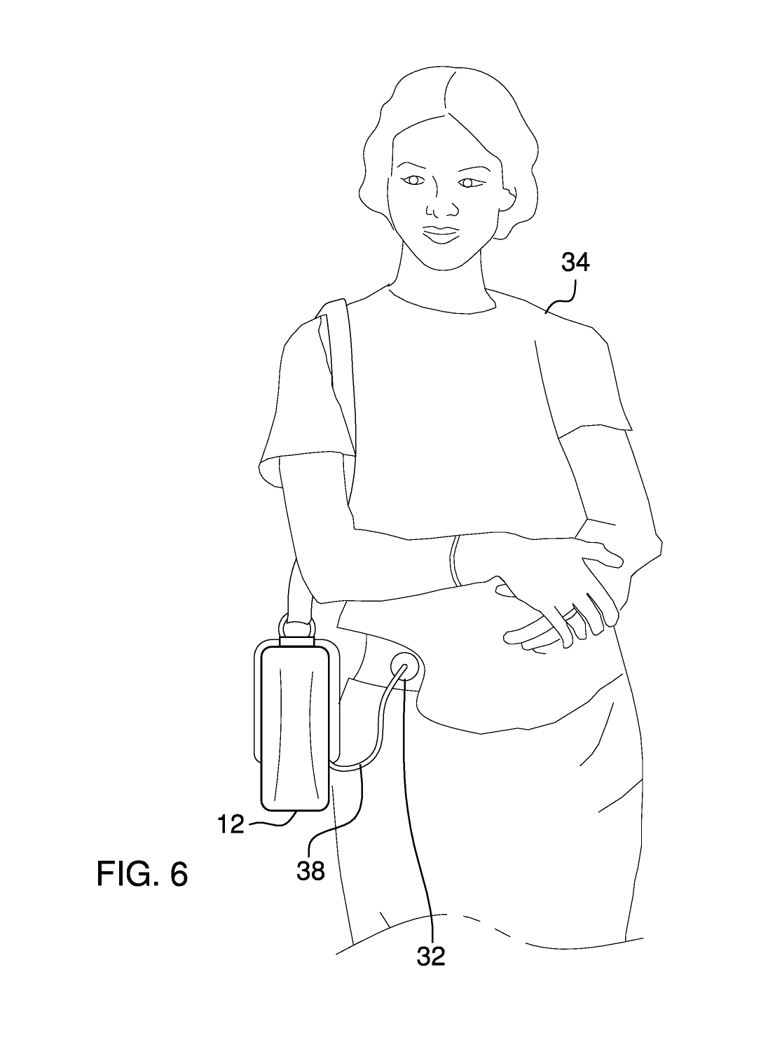

With reference now to the drawings, and in particular to FIGS. 1 through 6 thereof, a new urostomy bag device embodying the principles and concepts of an embodiment of the disclosure and generally designated by the reference numeral 10 will be described.

As best illustrated in FIGS. 1 through 6, the urostomy bag carrying system 10 generally comprises a bag 12 that may be carried. The bag 12 has an outer wall 14 and a pair of flaps 16. The outer wall 14 has a front side 18, a back side 20 and a top side 22. The top side 22 is open to access an interior of the bag 12. Each of the flaps 16 is coupled to the top side 22. Each of the flaps 16 is selectively draped downwardly along an associated one of the front side 18 and the back side 20. The bag 12 may be comprised of a fluid impermeable material such as vinyl or the like. Moreover, the bag 12 may be comprised of an opaque material.

The back side 20 has a pair of slots 24 extending into the interior of the bag 12. Each of the slots 24 has a leg 26 and a foot 28. The slots 24 are spaced apart from each other having the foot 28 corresponding to each of the slots 24 being directed toward each other. The leg 26 corresponding to each of the slots 24 intersects the top side 22 of the bag 12.

A strap 30 is provided. The strap 30 is coupled to the bag 12. The strap 30 may be slung over a shoulder. Thus, the bag 12 may be carried having the bag 12 positioned to cover a stoma 32 on user 34. The strap 30 may have an adjustable length.

A urostomy bag 36 is provided and the urostomy bag 36 is selectively positioned within the bag 12. At least one hose 38 is fluidly coupled to the urostomy bag 36. The at least one hose 38 is positioned in a selected one of the slots 24. The hose 38 extends outwardly from the foot 28 of the selected slot 24. Thus, the at least one hose 38 may be fluidly coupled to the stoma 32. The bag 12 is positioned to conceal the at least one hose 38. Thus, the bag 12 may enhance privacy of the user 34 with respect to the at least one hose 38 and the stoma 32.

A divider 35 may be provided and the divider 39 may be positioned within the bag 12. The divider 35 may be positioned between the front side 18 and the back side 20 of the bag 12. Thus, the divider 39 may define a first space 37 and a second space 39 in the interior of the bag 12. The urostomy bag 36 may be positioned in the second space 39. The first space 37 may contain any selected object commonly carried in a purse or the like.

A pair of tabs 40 is provided. Each of the tabs 40 is coupled to the back side 20 and each of the tabs 40 may be manipulated. Each of the tabs 40 is positioned adjacent to the leg 26 corresponding to an associated one of the slots 24. Each of the tabs 40 is selectively matable to the back side 20. Each of the tabs 40 has a distal end 42 with respect to the bag 12.

A pair of first fasteners 44 is provided. Each of the first fasteners 44 is coupled to an associated one of the tabs 40. Each of the first fasteners 44 is positioned adjacent to the distal end of the associated tab 40. A pair of second fasteners 46 is provided and each of the second fasteners 46 is coupled to the back side 20 of the bag 12. Each of the second fasteners 46 is positioned between the leg 26 corresponding to each of the slots 24. Each of the first fasteners 44 is complementary with an associated one of the second fasteners 46. Each of the first and second fasteners 46 may comprise a hook and loop fastener or the like.

In use, the urostomy bag 36 is positioned within the bag 12. The at least one hose 38 is slid downwardly along the leg 26 of the selected slot 24. The at least one hose 38 is further manipulated to be positioned on the foot 28 of the selected slot 24. A selected one of the tabs 40 is positioned to extend across the at least one hose 38 when the at least one hose 38 extends outwardly from the selected slot. Thus, the selected tab 40 secures the at least one hose 38 to the bag 12. An associated one of the flaps 16 is positioned to cover the back side 20 of the bag 12.

The at least one hose 38 is fluidly coupled to the stoma 32. Thus, the urostomy bag 36 collects waste fluid from the user 34. The bag 12 is positioned to cover the stoma 32 and the at least one hose 38 to enhance the user 34's privacy. The urostomy bag 36 is uncoupled from the stoma 32 and removed from the bag 12 when the urostomy bag 36 becomes full.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A urostomy bag carrying system comprising:
a bag being configured to be carried, said bag having a pair of slots extending into an interior of said bag;
a strap being coupled to said bag wherein said strap is configured to be slung over a shoulder thereby facilitating said bag to be carried having said bag being positioned to cover a stoma on user; and
a urostomy bag being selectively positioned within said bag, said urostomy bag having at least one hose being fluidly coupled thereto, said at least one hose being positioned in a selected one of said slots wherein said at least one hose is configured to be fluidly coupled to the stoma.

2. The system according to claim 1, wherein said bag has an outer wall and a pair of flaps, said outer wall having a front side, a back side and a top side, said top side being open to access an interior of said bag, each of said flaps being coupled to said top side, each of said flaps being selectively draped downwardly along an associated one of said front side and said back side, each of said slots extending through said back side, each of said slots having a leg and a foot, said slots being spaced apart from each other having said foot corresponding to each of said slots being directed toward each other, said leg corresponding to each of said slots intersecting said top side of said bag.

3. The system according to claim 2, wherein said bag is positioned to cover said at least one hose having said at least one hose being concealed wherein said bag is configured to enhance privacy of the user with respect to said at least one hose and the stoma.

4. The system according to claim 2, further comprising a pair of tabs, each of said tabs being coupled to said back side wherein each of said tabs is configured to be manipulated, each of said tabs being positioned adjacent to said leg corresponding to an associated one of said slots, each of said tabs being selectively matable to said back side, a selected one of said tabs being positioned to extend across said at least one hose when said at least one hose extends outwardly from said selected slot such that said selected tab secures said at least one hose to said bag.

5. A urostomy bag carrying system comprising:
a bag being configured to be carried, said bag having an outer wall and a pair of flaps, said outer wall having a front side, a back side and a top side, said top side being open to access an interior of said bag, each of said flaps being coupled to said top side, each of said flaps being selectively draped downwardly along an associated one of said front side and said back side, said back side having a pair of slots extending into said interior of said bag, each of said slots having a leg and a foot, said slots being spaced apart from each other having said foot corresponding to each of said slots being directed toward each other, said leg corresponding to each of said slots intersecting said top side of said bag;
a strap being coupled to said bag wherein said strap is configured to be slung over a shoulder thereby facilitating said bag to be carried having said bag being positioned to cover a stoma on user;
a urostomy bag being selectively positioned within said bag, said urostomy bag having at least one hose being fluidly coupled thereto, said at least one hose being positioned in a selected one of said slots having said hose extending outwardly from said foot of said selected slot wherein said at least one hose is configured to be fluidly coupled to the stoma, said bag being positioned to cover said at least one hose having said at least one hose being concealed wherein said bag is configured to enhance privacy of the user with respect to said at least one hose and the stoma; and
a pair of tabs, each of said tabs being coupled to said back side wherein each of said tabs is configured to be manipulated, each of said tabs being positioned adjacent to said leg corresponding to an associated one of said slots, each of said tabs being selectively matable to said back side, a selected one of said tabs being positioned to extend across said at least one hose when said at least one hose extends outwardly from said selected slot such that said selected tab secures said at least one hose to said bag.
